(19) United States
(12) Patent Application Publication
(10) Pub. No.: US 2011/0033477 A1
(43) Pub. Date: Feb. 10, 2011

(54) METHOD FOR DIAGNOSING AND TREATING CHRONIC PSYCHIATRIC ILLNESSES AND MARKERS AND TARGETS FOR SUCH METHODS

(75) Inventors: Carsten Korth, Dusseldorf (DE); Philipp Johannes Hendriks, Tonisvort (DE); Verian Bader, Recklinghuasen (DE); S. Rutger Leliveld, Neuss (DE)

Correspondence Address: RANKIN, HILL & CLARK LLP, 23755 LORAIN ROAD, SUITE 200, NORTH OLMSTED, OH, 44070, US

(73) Assignee: Carsten Korth, Dusseldorf (DE)

(21) Appl. No.: 12/934,639

(22) PCT Filed: Mar. 24, 2009
(86) PCT No.: PCT/EP09/02143

§ 371 Date: Oct. 21, 2010

(30) Foreign Application Priority Data
Mar. 26, 2008 (DE) 10 2008 016 064.4

(51) Int. Cl. A61K 39/395; C07K 16/18; G01N 33/566; C12Q 1/68; A61P 25/00

(52) U.S. Cl. 424/152.1; 530/388.2; 435/7.21; 435/6

(57) ABSTRACT

The invention relates to the use of special conformers of the DISC1 protein as a marker or target for chronic psychiatric illnesses, particularly schizophrenia, bipolar disorder, or depression.

The invention relates to markers and targets for the diagnosis and treatment of chronic psychiatric illnesses, in particular schizophrenia, bipolar disorder or depression, and to methods in which said markers or targets can be used.

Chronic psychiatric illnesses belong to the commonest diseases overall with lifetime prevalences of approx. 1% for schizophrenia and bipolar disorder and up to 10% for depression.

Little is known about the biological causes of these diseases. Neurotransmitter imbalances are responsible for certain phenotypes and symptoms of all these diseases and are used as a pharmacological target. How these neurotransmitter imbalances develop, however, i.e. the overriding causal processes leading to these disorders, is not known.

Diagnosis is similarly problematic. Up to now, diagnoses for these diseases have been made clinically by interview, as there are no biological markers suitable for routine diagnosis.

Genetic studies in families affected by chronic psychiatric illnesses have led to the identification of various genes that are probably associated with these diseases in a way that is still unknown. Because in these families one gene can have various phenotypes such as schizophrenia, bipolar disorder or recurring depression, it is presumed that—in contrast to existing concepts, based on purely clinical diagnosis—on the basis of new biological markers, there will be reclassification or re-definition in psychiatric diagnostics. A chronic psychiatric illness and in particular the resultant therapeutic strategy would then be defined on the basis of a biological marker and not a purely clinical diagnosis.

In some approaches, chronic psychiatric illnesses are diagnosed e.g. by measuring certain proteins or genetic markers.

In WO 2005/077978, a method is proposed, by which biological markers for chronic psychiatric illnesses such as schizophrenia, bipolar disorder and depression can be identified. In that method, in a first step a protein fraction is obtained from tissue samples from sick patients, in which insoluble proteins have accumulated. Using this protein fraction, antibodies are produced in the usual way, e.g. by immunization of animals. For antibodies that seem suitable, the proteins recognized by them are determined, so that these proteins, if they are present in insoluble form specifically only in sick patients, can be considered as markers.

Thus, a test is known, e.g. from U.S. Pat. No. 7,015,006, in which the activity or concentration of phospholipase (cPLA.sub.2) is measured. Increases of the measured values relative to normal values are taken as an indication for chronic psychiatric illnesses, e.g. schizophrenia.

U.S. Pat. No. 4,874,694 describes the measurement of phosphoprotein profiles by gel electrophoresis for diagnosing neurological diseases, e.g. schizophrenia.

US patent application 20050208519 relates to biomarker combinations, which can be used in the diagnosis of schizophrenia. These are certain genes and gene products, e.g. the genes of ADSS, APOBEC3B, (ATM); (CLC), CTBP1, C-X-C, (CXCL1), and others.

US patent application 2005/0089927 relates to special short peptides, which are bound selectively by samples from schizophrenic patients, whereas no binding or only slight binding occurs in samples from nonschizophrenic patients.

Other publications concern genes or protein markers that are correlated with DISC1 (Disrupted-In-Schizophrenia Protein 1) or DISC2. DISC1 is a gene that is translated to a protein, whose function is not yet known definitively. It interacts with various proteins that are associated with the cytoskeleton, and with factors that play a role in neuronal differentiation (review article in Chubb et al., 2008, Molecular Psychiatry 13:36). The binding of DISC1 to Nuclear Distribution Element 1 (NDEL1), Lis1 or PDE4 seems to have a special biological-functional role. DISC2 is a gene on the genomic antisense sequence of DISC1 (exon 8 and intron 9 of DISC1), which apparently is not translated into a protein; the function of this gene is still unknown.

WO 2004/071269 describes genetic markers and compositions for the diagnosis and treatment of neurological disorders. For this, the binding of DISC1 and FEZ1 (Fasciculation and Elongation Protein Zeta 1) is measured and a conclusion about the presence of a disorder is made based on the results.

WO 02/058637 relates to compositions and methods for diagnosis and treatment of neuropsychiatric disorders. New polymorphisms of DISC1 nucleic acid sequences and the DISC1 proteins encoded by them are described. The variant sequences or proteins on individual nucleotides can be correlated with neuropsychiatric disorders.

US application US 2005/0255500 relates to methods of diagnosis of psychiatric disorders, in which the molecular diversity or subcellular distribution of DISC1 is investigated and is correlated with pathological states.

None of the known markers or tests yet permits a reliable routine diagnosis of bipolar diseases, schizophrenia or depression or the re-definition of these and other chronic psychiatric illnesses mentioned above.

The task of the invention is to provide completely novel, DISC1-correlated markers or targets and use thereof in methods of diagnosis and treatment of neuropsychiatric disorders.

This task is solved with the markers or targets stated in the independent claims relating to applications and the methods of diagnosis or therapy stated in the independent method claims. Further independent claims are directed at antibodies, which can be used within the scope of the invention on the basis of their specific properties.

A feature common to all the independent claims is that they use DISC1-correlated conformers as markers, or targets for chronic psychiatric illnesses, in particular schizophrenia, bipolar disorders or depression.

The term conformers is intended to cover various DISC1 protein conformations including splice variants or degradation products as well as oligomers. Essentially, the DISC1-conformers mentioned according to the invention are proteins that are sarcosyl-insoluble in ultracentrifugation conditions. For example, shortened DISC1 proteins, which arise either through alternative splicing (transcripts) or protease degradation (degradation/cleavage products), have an increased insolubility or aggregation tendency, since certain subdomains that are conducive to aggregation are strongly exposed within DISC1. It is therefore possible that certain shortened DISC1 proteins aggregate “automatically”; just the detection of these shortened forms (e.g. with the antibody mAB 3D4.F3 mentioned hereunder) can therefore already be a positive test according to the invention even in assays that do not yet investigate the solubility of the conformers directly.

Essential aspects of the invention relate, as mentioned above, to the fact that in chronic psychiatric illnesses DISC1 has a tendency to multimerization. One aspect of the invention therefore relates to the use of said DISC1-conformers as markers, or targets, which have an increased aggregation propensity, i.e. an increased tendency to form insoluble multimers in particular. Another aspect relates to the multimers themselves, which can also be used as markers, or targets.

Furthermore, the loss of certain functionalities of the DISC1 protein is also used as a disease-specific marker or target.

Demarcation of the stated markers or targets against other DISC-conformers was effected according to the invention by means of antibodies that selectively recognize the stated disease-specific conformers of the DISC1 protein.

These are the monoclonal antibodies mAB 3D4.F3 and mAB 19F7.A9 that have been isolated by the applicant.

The antibody mAB 3D4.F3 is secreted by a hybridoma cell line deposited by the applicant on the 12 Mar. 2008 under number DSM ACC2899 at the DSMZ-Deutsche Sammlung von Mikroorganismen und Zellkulturen GmbH (German collection of microorganisms and cell cultures).

The antibody mAB 19F7.A9 is secreted by a hybridoma cell line deposited by the applicant on the 12 Mar. 2008 under number DSM ACC2900 at the DSMZ-Deutsche Sammlung von Mikroorganismen und Zellkulturen GmbH.

The antibodies were produced by immunizing mice, which have the genetic 129/SvEv background, with recombinant DISC1 fragments, which were expressed in E. coli. It was shown for the 129/SvEv mouse strain that they have a genetic defect, which modifies the expression of the DISC1 protein (Koike et al., 2006, PNAS 103:3693), so that an improved immune response to the DISC1 protein is possible. Two soluble protein fragments of DISC1 were cloned and expressed in E. coli: DISC1 598-854 (DISC598), and DISC1 316-854 (DISC316). mAB 19F7.A9 was found after immunization with DISC 598, mAB 3D4.F3 after immunization with DISC316.

It is envisaged according to the invention that a degradation product recognized by the monoclonal antibody mAB 3D4.F3 or a DISC1 protein corresponding to a special transcript of the DISC1 protein is used as marker or target for chronic psychiatric illnesses.

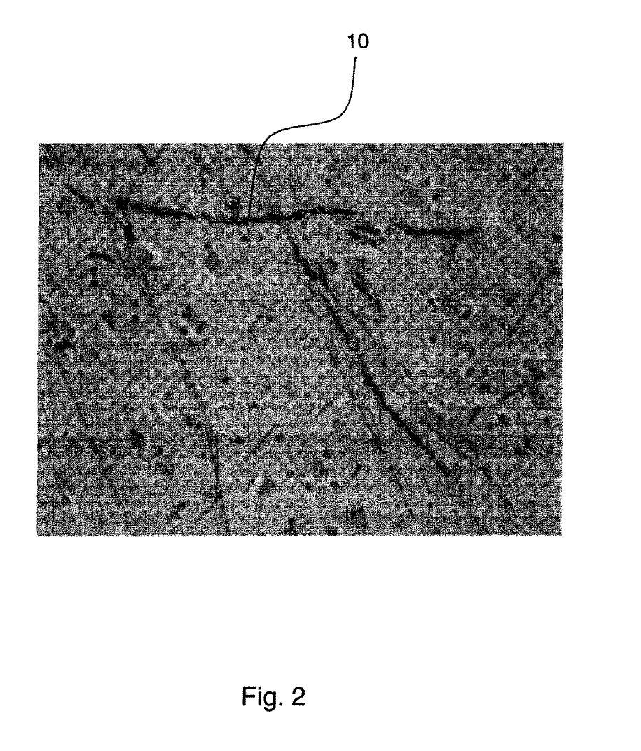

It makes no difference whether full length DISC1, shorter degradation/cleavage products of DISC1 or DISC1 proteins corresponding to special transcripts aggregate. It is, however, probable (FIG. 3) that shorter DISC1-derived polypeptide sequences possess a higher aggregation propensity, i.e. aggregate more readily and then lead to cellular malfunctions, which finally can give rise to disease symptoms of the chronic psychiatric type. In the human brain these appear to be products that preferably are separated electrophoretically at approx. 70-75 kDa, and 55 kDa. These shorter polypeptide fragments, which react with mAB 3D4.F3, can be degradation/cleavage products, DISC1 proteins corresponding to special transcripts, or even both.

The DISC1 protein or splice forms thereof can be cleaved by endogenous proteases specifically or nonspecifically to shorter fragments, and these forms then have biologically novel properties. Certain forms then have a greater tendency to aggregate as insoluble complexes.

The DISC1 gene has 13 extrons, so that theoretically a large number of different transcripts is conceivable, which can be expressed in different splice variants. In addition, degradation or cleavage products can theoretically form at many sites. These can only be specified more precisely once the proteases are found that degrade DISC1 or splice variants thereof specifically and/or nonspecifically. In this sense it can also be predicted that the activity of DISC1-cleaving proteases can lead indirectly to a DISC1 pathology and that mutations in these proteases, which lead to increased cleavage, can also be associated with chronic psychiatric illnesses.

It is known from WO 02/058637 that special antibodies that may be present can bind to various DISC1-amino acid sequences that are correlated with psychiatric illnesses, which are said to correspond to specific polymorphisms (these polymorphisms are listed in Table 6 in WO 02/058637). WO 02/058637 does not deal with specific transcripts or splice variants, but with allele variants due to polymorphisms that are associated with disease symptoms of various neuropsychiatric illnesses. There is also mention of antibodies, especially monoclonal antibodies that bind to a sequence that represents the full length DISC1 protein. Thus, there is no presentation of splice variants that have a shorter length, nor is a specific antibody deposited, or a specific epitope described that is associated with a disease and is expressed differentially.

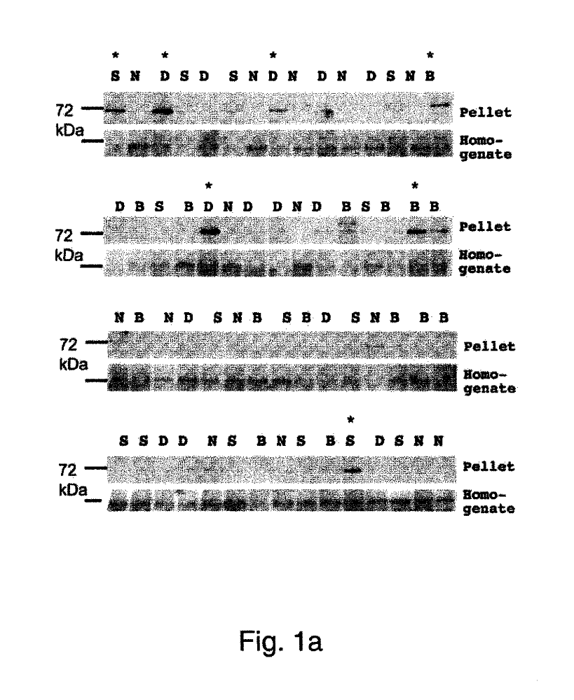

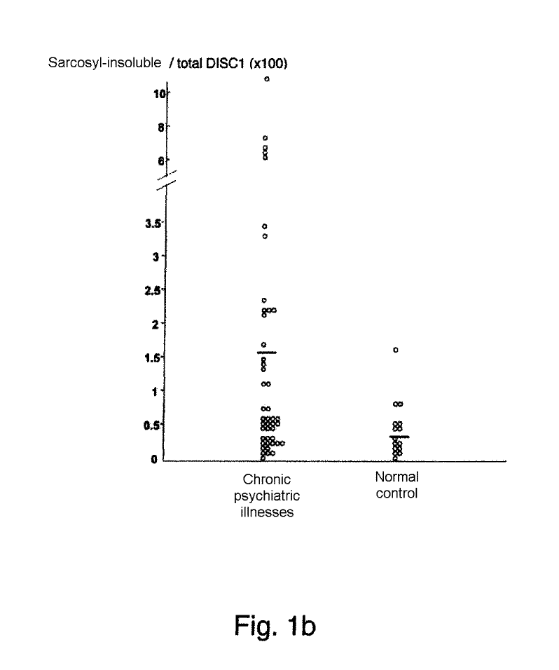

The antibody 3D4.F3 used here according to the invention is now, as shown in FIG. 1 or FIG. 2, able to selectively stain certain cellular and local brain structures in brain sections from sick patients and furthermore shows, in Western blotting, an extremely strong reaction in some individuals with chronic psychiatric illnesses. Moreover, owing to the heterogeneity of the chronic psychiatric illnesses, it is clear that not all individuals with these diagnoses have corresponding pathology of the DISC1 protein, i.e. aggregated forms. Rather, it is probable that this relates to a subgroup of these patients, who however (according to current clinical criteria) are phenotypically different, i.e. have the diagnoses schizophrenia, bipolar disorder, or depression.

The antibody mAB 3D4.F3 represents an advantage over antibodies known until now. As far as the applicant knows, it is the first and so far only known antibody that is able:

1. to stain individual disease-specific axons and dendrites in the transition zone from the white to the gray matter in convolutions (gyri) of the cortex, and this axonal and dendritic staining has indications of a contorted, tangled bundle. An overall view of various neuropathological criteria on the 3D4.F3-stained brain section makes it possible to differentiate sick from healthy individuals.

2. to recognize a special splice or degradation form in a group of patients with chronic psychiatric illnesses protein-biochemically in the unfractionated brain homogenate, which separates electrophoretically at approx. 70-75 kDa (FIG. 2).

Up to now, it was not known in the prior art that DISC1 proteins corresponding to special transcripts or degradation/cleavage forms of DISC1 were associated with phenotypes of chronic psychiatric illnesses such as schizophrenia, bipolar disorder, or depression. Also, no specific neuropathology of DISC1 had been described. The present mAB 3D4.F3 can, however, show this for the first time.

Alternatively, it is envisaged according to the invention that an insoluble multimer of the DISC1 protein that is recognized by the monoclonal antibody mAB 19F7.A9 is used as marker or target for chronic psychiatric illnesses. The applicant was able to demonstrate, in post-mortem brain samples from a subgroup of patients with neuropsychiatric illnesses, that significant proportions of the DISC1 protein occur as protein aggregates that are insoluble in cold sarcosyl. These aggregates could not be detected in the healthy control samples. Sarcosyl-insoluble protein aggregates can be detected by any DISC1-specific antibodies, e.g. also the polyclonal antisera stated in FIG. 1.

The antibody mAB 19F7.A9, in contrast, is able, as the applicant showed with the Dot Blots reproduced in FIG. 3, to bind, without special biochemical purification based on solubility, specifically to insoluble oligomers of the recombinant DISC598 protein, and does not bind to the soluble dimers. The antibody thus binds specifically to disease-associated aggregates of the DISC1 protein.

The invention relates furthermore to methods of diagnosis of chronic psychiatric illnesses, which use DISC1 proteins corresponding to special transcripts, degradation/cleavage products, aggregates or functionalities of DISC1 as disease-specific markers.

In a method according to the invention, a tissue sample obtained from a study subject to be investigated is analyzed for whether a disease-specific degradation or cleavage form is present in excessive quantity in the sample or if it contains DISC1 proteins corresponding to special transcripts of the DISC1 protein, the presence of said degradation or cleavage form or of a DISC1 protein corresponding to a special transcript being indicative of the presence of a chronic psychiatric illness.

In particular the method is an immunoassay, in which a DISC1 protein corresponding to a special transcript or degradation product-specific antibody, e.g. the aforementioned mAB 3D4.F3, is used.

It is also conceivable to investigate the presence of defined, disease-specific transcripts, which correspond to the aggregation-favorable DISC1 proteins, at the mRNA level, for instance by analysis of suitable samples by RNA-preparing methods, for instance the polymerase chain reaction after reverse transcription or Northern blots.

In alternative methods according to the invention for the diagnosis of chronic psychiatric illnesses, the insoluble variant of DISC1, which is probably related to aggregation or multimerization of the DISC1 protein that is correlated with the disease, is used intentionally as a marker. As certain degradation/cleavage products of DISC1, or DISC1 proteins corresponding to special transcripts are especially aggregation-promoting, under certain circumstances it is possible in some cases to carry out investigations that comprise both a determination of reactivity with 3D4.F3 (for the presence of particular degradation forms or DISC1 proteins corresponding to special transcripts) and a determination of reactivity with 19C3.A9 in the native state. This further advantageous embodiment relates to a method in which several of the DISC1-correlated disease-specific markers mentioned up to now are investigated simultaneously. This can take place e.g. with an immunoassay, in which the antibodies mAB 19F7.A9 and mAB 3D4.F3 are used simultaneously. The advantage is that a disease can be recognized more reliably in different stages.

In another embodiment of the method it is envisaged that the tissue used as sample material is purified biochemically in such a way that the insoluble DISC1 form, if present, is enriched and can then be detected with an antibody that generally binds DISC1, e.g. the polyclonal sera mentioned in FIG. 1.

Preparation was carried out as described in WO 2005/077978. This is a usual protocol, as used routinely for enrichment of insoluble proteins in tissue samples. Therefore no details of the protocol are given. Other protocols known by a person skilled in the art can of course also be used.

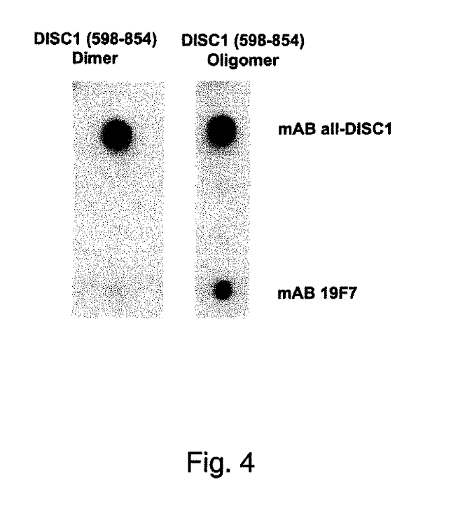

Another advantageous embodiment of the invention envisages that at least one of the DISC1-correlated markers mentioned so far is used together with at least one other marker for the diagnosis of chronic psychiatric illnesses. Conceivable other markers are e.g. the insoluble variants of the protein CRMP1 and proteins that are recognized by an antibody described by the applicant in DE 102007022669.3 and deposited on 4 Apr. 2007 under number DSM ACC2836 with the designation 6H11 and/or by an antibody described in WO 2005/077978 and deposited on 26 Jan. 2005 under number DSM ACC2713 with the designation 7B2, to mention just a few examples. For these investigations it would be necessary, if no conformation-specific antibodies are used, first to purify the insoluble proteome biochemically as described in WO2005/077978. FIG. 6 shows how such a combination of detection of various proteins in the insoluble proteome increases the resolution for differentiating between sick and healthy individuals and can also define other disease groups that are not associated with insoluble DISC1.

In this connection, an immunoassay would be preferable, in which several antibodies, e.g. the aforementioned antibodies, are used simultaneously, and bind specifically to the markers. It is, however, also conceivable that the markers, after preparation of the sample that promotes the formation of the disease-specific markers, are detected by means of antibodies that bind the denaturing form.

These methods can also be applied advantageously in tests in which polypeptides defined by mAB 3D4.F3, which can be certain degradation/cleavage products or transcripts of DISC1, can be detected in body fluids or tissue samples. Thus, it is conceivable, for example, that 3D4.F3 could be used for a blood test, in which immunoreactivity can be detected in the plasma, in the serum, or specified fractions of white blood cells. The same could be done with spinal fluid, tissue biopsies, such as skin, muscle or nerve biopsy. It is also conceivable that 3D4.F3 is immunoreactive with DISC1 in lacrimal fluid, in saliva or in urine, and that this immunoreactivity is defined by a particular pathology, which can lead finally to a chronic psychiatric illness.

Within the scope of detection of DISC1, it may be necessary to purify DISC1 from tissues, tissue homogenates, or tissue fluids, in particular in order to concentrate those DISC1 degradation/cleavage forms or DISC1 proteins corresponding to special transcripts, that are only present in small amounts. It is envisaged according to the invention that immobilized p-aminobenzamidine is used as binding matrix e.g. in columns, for enriching DISC1 from fluids. DISC1 protein enriched in this way according to the invention can then optionally be further fractionated or further processed.

Another variant of a method according to the invention is based on the fact that insoluble, and thus as a rule disease-specific, forms of the DISC1 protein in particular show greatly reduced interaction with ligands. The applicant was able to show that exclusively octamers of recombinant DISC1 (598-854) bind NDEL1, but not dimers or high-molecular multimers of DISC1 (598-854). This reduced binding capacity of DISC1 (598-854) can also be assessed as a disease-specific indicator and used as a marker in a diagnostic method.

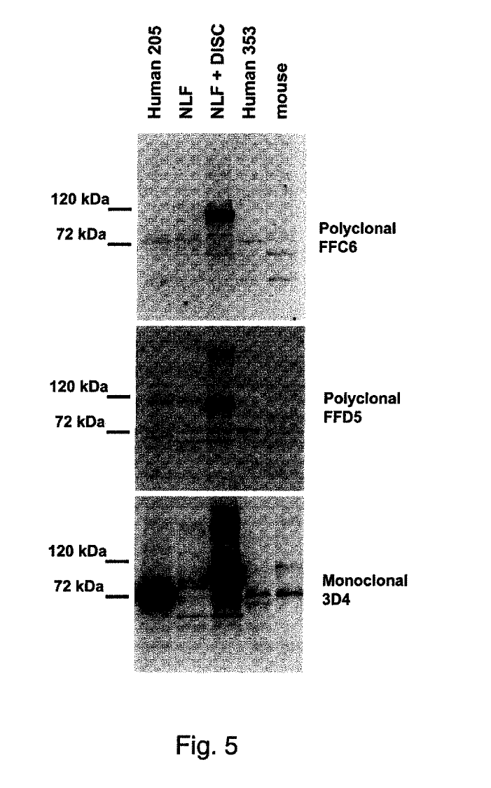

For example, the presence of native, soluble low-molecular NDEL1 protein (approx. 43-90 kDa) can be investigated in a tissue sample in question. If no, or only physiologically little, insoluble DISC1 protein is present, low-molecular NDEL1 protein should also be detected. If, however, insoluble DISC1 protein is present, the soluble, low-molecular NDEL1 protein disappears. This relationship is shown in FIG. 7. The conformers described or properties of the DISC1 protein can of course not only be used as markers in diagnostic techniques. It is very probable that e.g. the aggregation of the DISC1 protein or the associated loss of function is linked causally to the development of the diseases.

Therefore according to the invention it is further envisaged that DISC1 proteins corresponding to special transcripts or degradation/cleavage products, insoluble aggregates of the DISC1 protein and the reduced binding property can be used as therapeutic targets.

It is conceivable, for example, to use these conformers or properties of the DISC1 protein for screening for substances that for example suppress the formation of the disease-specific transcripts or reverse it (aggregates) or restore the binding capacity of DISC1 proteins with reduced capacity for interaction. Substances with the desired properties can then be used for treating patients.

Within the scope of said screening for suitable substances, for example cell lines can be transfected with DISC1 and then the transfected cell lines can be incubated with the test substances. Then within the scope of biochemical purification, insoluble DISC1 is isolated from the cell lines. The proportion of insoluble DISC1 is then compared with that in untreated control cell lines.

The invention will be explained in more detail below, on the basis of the drawings.

FIG. 1a shows a comparison of Western blots for DISC1 from sarcosyl-insoluble pellets (P; top) and the initial brain homogenate (H; bottom) from sixty different cases. (S) stands for schizophrenia, (B) for biopolar disorder, (D) for depression and (N) is the control.

FIG. 1b is a scatter plot of the quantitative ratio of the gray value of pellets to that of the homogenate in the region of the 72 kD band. Only cases with chronic psychiatric illness were compared with normal diagnoses, since the “DISC1-positive” cases comprise all three diagnoses schizophrenia, bipolar disorder and depression.

It can be seen from FIG. 1a that not all patient samples diagnosed as sick show a band that is correlated with the presence of insoluble DISC1. This is presumably connected with the fact that the “insoluble DISC1 positive” cases only represent a subgroup of these diseases. The applicant is of the opinion that the patients classified e.g. as schizophrenic or depressive are in fact a heterogeneous group of different clinical pictures, of which perhaps only one correlates with the DISC1-marker. This could be why insoluble DISC1 is only detectable for some of the patients classified as e.g. schizophrenic/depressive. It is to be assumed that for example the use according to the invention of new biological markers will lead to re-classification or a re-definition in psychiatric diagnostics. The result may be a new doctrine, to the effect that psychiatric illnesses are no longer defined by means of clinical diagnoses, but on the basis of special biological markers.

The results seen in FIG. 1a are confirmed by FIG. 1b. According to this, 86% of the positive cases from FIG. 1a also have a high P/H ratio. The mean values for samples from patients diagnosed as sick are, at 1.6, well above the value of 0.4 for healthy patients (P 0.002 in an analysis of variance [ANOVA]).

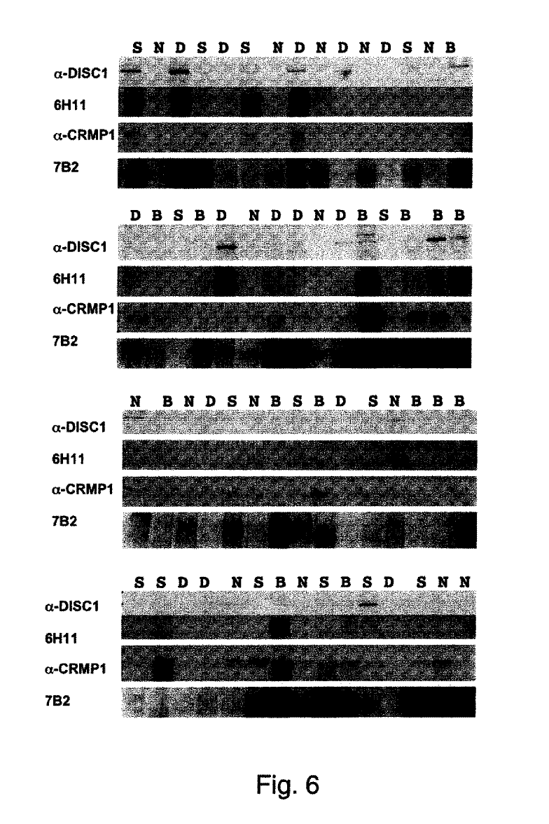

FIG. 2 shows a brain section, stained with mAB 3D4.F3, from a patient with depression. It can be seen that the antibody selectively stains certain axons or dendrites in the cortex, marked with the FIG. 10, and at the same time only stains nerve cells weakly, or not at all.

FIGS. 3a and b show Western blots of fractionated lysates of human neuroblastoma cells (NLF), which express DISC1 recombinantly, after they were transfected with a plasmid that codes for full length DISC1 (D14=for 14 hours, D24=for 24 hours) or were transfected with a plasmid that codes for full length NDEL1 (N14=for 14 hours, N24=for 24 hours).

FIG. 3a shows the soluble fraction (supernatant) after low-level centrifugation; it can be seen that the expression of DISC1 (D14, D24), or NDEL1 (N14, N24) increases as a function of time.

FIG. 3b shows the sarcosyl-insoluble fraction (pellet) after ultracentrifugation in sarcosyl buffer. It can be seen that in the case of DISC1 overexpression, but not in the case of NDEL1 overexpression, insoluble protein accumulates, and degradation products of DISC1 are precipitated disproportionally at 70-75 kDa and 55 kDa (marked with a star). It can also be seen that insoluble DISC1 can no longer bind NDEL1, as otherwise it would entrain NDEL1 into the pellet.

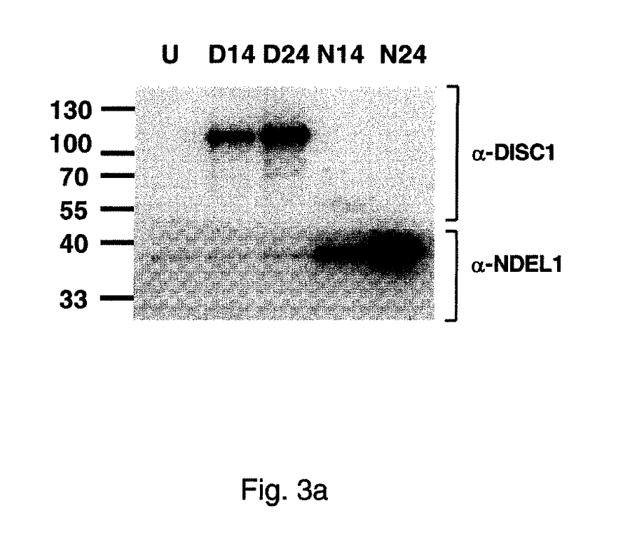

FIG. 4 shows Dot Blots of mAB 19F7.A9 and an antibody mAB all-DISC1 that recognizes non-conformation-specific DISC1 protein. The reactivity of the antibodies was tested with dimers of recombinant DISC1 (598-854) protein, which was produced in E. coli and simulates the behavior of full length DISC1 protein from the brain (picture on left), and oligomers of the recombinant DISC (598-854) protein that was also produced. It can be seen that the nonspecific DISC1-protein-recognizing antibody recognizes both dimers and oligomers. In contrast, the antibody mAB 19F7.A9 only recognizes oligomers.

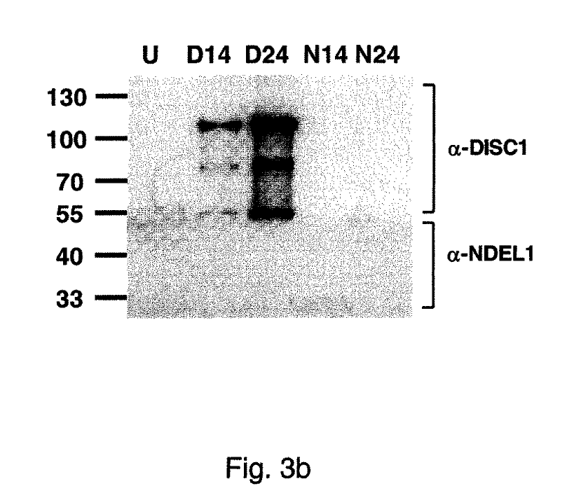

FIG. 5 shows, in Western blots, the immunoreactivity of the antibody 3D4.F3 (bottom picture) in brain homogenates from patients with sporadic psychiatric illnesses compared with the polyclonal sera FFC6 (top picture) and FFD5 (middle picture).

In the first band, disease-associated brain homogenate (Human 205) was applied in all three experiments. The second lane contains the lysate of a human neuroblastoma cell line designated NLF. The third lane contains NLF+full length DISC1 protein recombinantly in this. The fourth band contains (Human 353) brain homogenate from a healthy patient. Finally, the fifth lane is brain homogenate from a mouse.

It can be seen that the polyclonal sera FFC6 and FFD5 and the antibody 3D4.F3 bind to the recombinant DISC1 protein. Additionally the antibody 3D4.F3 binds apparently with exceptionally high affinity to brain homogenate of the sick patient (Human 205), but not to the homogenate from a healthy subject (Human 353). This indicates that the brain homogenate of the sick patient contains a disease-associated degradation form or DISC1 proteins corresponding to special transcripts, which cannot be found in this quantity in healthy brain homogenates.

FIG. 6 shows an array of Western blots. It shows the investigations of insoluble protein fractions from 60 post-mortem brains (corresponding to those in FIG. 1a), with staining with 4 different antibodies: a polyclonal antiserum to DISC1, which specifically recognizes DISC1 (as in FIG. 1a), the aforementioned antibody designated mAB 6H11, which cross-reacts with CRMP1, a commercially available (ProSCi 3625; from the company ProSci, Poway, USA; catalog number 3625) anti-CRMP1 antibody (a-CRMP1) and the aforementioned monoclonal antibody 7B2. It can be seen that the combined staining of the insoluble protein fraction from brains with different antibodies, which recognize schizophrenia-associated insoluble proteins, facilitates the resolution in the identification of diseased brains. Moreover, it becomes clear that some diseased brains are associated with different insoluble proteins, which was to be expected based on the biological heterogeneity of these diseases.

Information concerning deposition: according to the Budapest Treaty

A hybridoma cell line, which secretes the antibody mAB 3D4.F3, was deposited on 12 Mar. 2008 under number DSM ACC2899 at the DSMZ-Deutsche Sammlung von Mikroorganismen und Zellkulturen GmbH, Inhoffenstrasse 7B, 38124 Braunschweig. A hybridoma cell line, which secretes the antibody mAB 19F7.A9, was deposited on 12 Mar. 2008 under number DSM ACC2900 at the DSMZ-Deutsche Sammlung von Mikroorganismen und Zellkulturen GmbH, Inhoffenstrasse 7B, 38124 Braunschweig.

1-5. (canceled)

6. A monoclonal antibody mAB 3D4.F3 to DISC1 secreted by hybridoma cell line DSM ACC2899.

7. A monoclonal antibody mAB 19F7.A9 to DISC1 aggregates secreted by hybridoma cell line DSM ACC2900.

8. A method for the diagnosis of chronic psychiatric illnesses, the method comprising analyzing a tissue sample obtained from a study subject for whether the sample contains a disease-specific DISC1-degradation/cleavage product or DISC1 proteins corresponding to special transcripts, wherein the presence of said product or transcript indicates the presence of a chronic psychiatric illness.

9. The method as claimed in claim 8, wherein an immunoassay in which a specific antibody for the disease-specific degradation/cleavage product or in which a DISC1 protein corresponding to a special transcript is used.

10. The method as claimed in claim 9, wherein the specific antibody is monoclonal antibody mAB 3D4.F3.

11. A method for the diagnosis of chronic psychiatric illnesses, the method comprising analyzing a tissue sample obtained from a study subject RT-PCR or Northern blot for whether a disease-specific transcript of DISC1 or DISC2 is present in the sample.

12. A method for the diagnosis of chronic psychiatric illnesses, the method comprising analyzing a tissue sample obtained from a study subject for whether DISC1 protein contained in the sample is present as a multimer, wherein the presence of DISC1 protein multimers indicates the presence of a chronic psychiatric illness.

13. The method as claimed in claim 12, wherein an immunoassay in which an antibody specifically binding the multimerized form of the DISC1 protein is used.

14. The method as claimed in claim 13, wherein the specific antibody is monoclonal antibody mAB 19F7.A9.

15. A method for the diagnosis of chronic psychiatric illnesses, the method comprising analysing by an immunoassay a tissue sample obtained from a study subject in which both antibody mAB 19F.7A9 and antibody mAB 3D4.F3 are used.

16. A method for the diagnosis of chronic psychiatric illnesses, the method comprising analysing a tissue sample obtained from a study subject simultaneously for the presence of:
at least one of the DISC1-correlated disease-specific markers selected from the group of a conformer of the DISC1 protein recognized by the monoclonal antibody 3D4.F3 and a conformer of the DISC1 protein recognized by the monoclonal antibody mAB 19F7.A9; and
at least one further marker for chronic psychiatric illnesses.

17. The method as claimed in claim 16, wherein the at least one further marker is an insoluble variant of protein CRMP1, an antigen recognized by antibody 6H11 or an antigen recognized by antibody 7B2.

18. The method as claimed in claim 12, wherein the tissue sample is processed according to a protocol in which insoluble proteins are enriched in a fraction, the fraction containing enriched insoluble proteins is incubated with a DISC1-specific antibody, and any binding of the DISC1-specific antibody to proteins contained in the fraction is taken as an indication of the presence of a chronic psychiatric illness.

19. A method for the diagnosis of chronic psychiatric illnesses, the method comprising analyzing a tissue sample obtained from a study subject for the capacity of DISC1 protein contained in the sample for the binding of NDEL1, wherein at least partial inability of the DISC1 protein contained in the sample to bind NDEL1 is taken as an indication for the presence of a chronic psychiatric illness.

20. The method as claimed in 8, an immobilized p-aminobenzamidine or its derivatives is used to enrich DISC1 protein.

21. A method for the treatment of chronic psychiatric illnesses, comprising administering to a patient a substance that is able to prevent, slow down or reverse aggregation of the DISC1 protein.

22. A method for the treatment of chronic psychiatric illnesses, comprising administering to a patient a substance that is able to inhibit protease activity that leads to a disease-specific degradation or cleavage product of DISC1.

23. A method for the treatment of chronic psychiatric illnesses, comprising administering to a patient a substance that is able to prevent transcription of the DISC1 or DISC2 gene that leads to a disease-specific transcript.

24. A method for the identification of a substance that is able to:
prevent, slow down or reverse aggregation of the DISC1 protein;
inhibit protease activity that leads to a disease-specific degradation or cleavage product of DISC1; or
prevent transcription of the DISC1 or DISC2 gene that leads to a disease-specific transcript;
the method comprising transfecting a cell line with DISC1, incubating the transfected cell line with a test substance, and comparing the proportion of insoluble DISC1 in the cell line incubated with the test substance with the proportion of insoluble DISC1 in a cell line that was not incubated with the test substance.

25. The method as claimed in claim 24, wherein mAB 3D4.F3 is used as the test substance.
